(19) United States
(12) Patent Application Publication
(10) Pub. No.: US 2018/0328885 A1
(43) Pub. Date: Nov. 15, 2018

(54) COMBINED AND FREE CHLORINE MEASUREMENT THROUGH ELECTROCHEMICAL MICROSENSORS

(71) Applicant: HACH COMPANY, Loveland, CO (US)

(72) Inventors: Vishnu Rajasekharan, Fort Collins, CO (US); Corey Salzer, Fort Collins, CO (US)

(21) Appl. No.: 15/775,928

(22) PCT Filed: Nov. 11, 2016
(86) PCT No.: PCT/US2016/061602

§ 371 Date: May 14, 2018

Related U.S. Application Data
(60) Provisional application No. 62/254,379, filed on Nov. 12, 2015.

(51) Int. Cl. G01N 27/416; G01N 33/18; G01N 27/403

(52) U.S. Cl. CPC G01N 27/4168; C02F 2209/29; G01N 27/403; G01N 33/18

(57) ABSTRACT

One embodiment provides a method, including: initiating, at a generator electrode in an electrode array having a collector electrode adjacent to but physically separate from the generator electrode, a reduction reaction for an oxygen containing species and a monochloramine species present in a water sample; said initiating comprising generating, at the generator electrode, a generator current producing the reduction reaction; detecting, at the collector electrode, a collector current associated with products formed from the reduction reaction; wherein the electrode array is biased to preferentially detect one or more products of the reduction reaction; and determining, by comparing the generator current with the collector current, concentrations of oxygen containing species and monochloramine species present in the water sample.

### FIELD

The subject matter described herein relates to the general field of water quality measurement. More specifically, the subject matter relates to monitoring chlorine and monochloramine levels by using new electrochemical sensor designs and methods.

### BACKGROUND

The determination and monitoring of chlorine residuals in water is an important analytical process for ensuring the safety of water for human consumption and for the environment. Analyses of disinfectant species are critical to maintain the minimum residual level at the drinking water treatment plant and in the drinking water distribution systems. Several methods are commercially available for the measure of residual chlorine in water. Commercially available methods range from simple test strips, to colorimetric tests, to electrochemical sensors utilizing amperometric methods.

### BRIEF SUMMARY

In summary, one aspect provides a method, comprising: initiating, at a generator electrode in an electrode array having a collector electrode adjacent to but physically separate from the generator electrode, a reduction reaction for an oxygen containing species and a monochloramine species present in a water sample; said initiating comprising generating, at the generator electrode, a generator current producing the reduction reaction; detecting, at the collector electrode, a collector current associated with products formed from the reduction reaction; wherein the electrode array is biased to preferentially detect one or more products of the reduction reaction; and determining, by comparing the generator current with the collector current, concentrations of oxygen containing species and monochloramine species present in the water sample.

Another aspect provides an apparatus, comprising: an electrode array, comprising: a generator electrode; and a collector electrode; said electrode array having the collector electrode disposed adjacent to but physically separate from the generator electrode; the generator electrode providing a current that initiates a reduction reaction for an oxygen containing species and a monochloramine species present in a water sample; the collector electrode detecting a collector current associated with products formed from the reduction reaction; wherein the electrode array is biased to preferentially detect one or more products of the reduction reaction; and a processor that determines, by comparing the generator current with the collector current, concentrations of oxygen containing species and monochloramine species present in the water sample.

A further aspect provides a chlorine probe, comprising: an electrode array, comprising: a metal generator electrode; and a metal collector electrode; said electrode array having the collector electrode and the generator electrode deposited as nanostructures adjacent to, but physically separate from, one another; the generator electrode: providing a current that initiates a reduction reaction for an oxygen containing species and a monochloramine species present in a water sample; the collector electrode detecting a collector current associated with products, byproducts or intermediate species formed from the reduction reaction; wherein the electrode array is biased to preferentially detect the one or more products byproducts or intermediate species of the reduction reaction by use of a biasing mechanism selected from the group consisting of: a thin layer of material deposited between the metal generator electrode and the metal collector electrode that preferentially absorbs or stabilizes one or more of the products, byproducts or intermediate species; and a pH control element that generates protons between the metal generator electrode and the metal collector electrode; and a processor that determines, by comparing the generator current with the collector current, concentrations of oxygen containing species and monochloramine species present in the water sample.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE SEVERAL VIEWS OF THE DRAWINGS

FIG. 1 illustrates an example schematic of oxygen and monochloramine determinations made with generator-collector systems.

FIG. 2 showing a generator electrode (GE1) and a collector electrode (CE1) couple for direct detection method.

FIG. 3 illustrates an example schematic showing a generator electrode (GE1) and a collector electrode (CE1) couple for a space layer method.

FIG. 4 illustrates an example schematic showing a generator electrode (GE1) and a collector electrode (CE1) couple for a pH controlled method.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

Currently the predominant disinfectants in drinking water are chlorine based (free chlorine and combined chlorine). The standard methods for analyses of such disinfectants in drinking water are accomplished by colorimetric methods utilizing redox active dyes. A commonly expressed drawback to the colorimetric methods is the need for maintaining instrumentation and reagents. The cost of ownership for such instruments, particularly those in remote locations, can be significant.

Alternative methods, including electrochemical approaches, have been undertaken to develop a reagent-less system that has a lower cost of ownership. Membrane-based amperometric sensors are available for measurement of free and total chlorine measurements. However, such sensors are not sufficiently robust for in-pipe applications, are prone to fouling, are sensitive to flow, pressure, and pH fluctuations. Membrane-less amperometric sensors are also sensitive to flow and are even more sensitive to pH than the membrane-based probes. Additionally, membrane-less probes have not yet been able to satisfactorily measure total chlorine or monochloramine because of persistent interference from dissolved oxygen. While amperometric chlorine sensors have a commercial presence in the drinking water markets, they do not yet meet desires and expectation of most customers.

Advancements in microfabrication methods and lithographic techniques have enabled novel electrode architectures, including dual electrode arrangements comprising generator-collector systems fabricated with high precision having micro- and nano-scale dimensions. In such systems, parallel, coplanar electrodes (metal, metal oxide, or any other conductive or non-conductive substrates) are positioned in solution containing the analyte of interest. The electrodes can be operated in either flowing or static sample conditions.

In the simplest form, two electrodes are employed. One of the electrodes serves as a generator electrode. In a flowing system, this is commonly placed “upstream” from the second electrode. This electrode is held at, or scanned past, a potential that corresponds to the oxidation or reduction potential of an analyte of interest. The second electrode is called the collector. If placed “downstream” from the generator electrode, the collector electrode can monitor changes in oxidation or reduction of electroactive species generated or consumed as a function of the initial redox process at the generator electrode. The generator-collector electrodes can be in nanometer dimensions for spacing and width of the electrodes optimize the collection efficiency for the generator-collector electrode couple.

The pH in the vicinity of the electrode system may be controlled by the generation of protons or hydroxide ions via electrochemical methods, e.g., two electrodes positioned in close proximity to a set of generator and collector electrodes. The potential of these electrodes can be set to a value at which protons can be generated via the reduction of water. Using nano-electrodes can provide for precise control of localized pH, enabling the ability to finely control the localized pH around the vicinity of the generator and collector electrodes. This can be used to detect monochloramine accurately.

Accordingly, an embodiment provides a method for distinguishing and measuring oxygen and monochloramine present in a sample. In an embodiment, the interference by oxygen can be accounted for by monitoring for intermediates, products, or other changes in the redox conditions at a collector electrode as a function of the processes occurring at a generator electrode. In an embodiment, a generator-collector electrode system can be used to differentiate the co-reduction current from monochloramine and oxygen. These two species are known to undergo electrochemical reduction at nearly the same potentials. The irreversible reactions that occur at the electrodes is monitored. In an embodiment, the analyte and interferants may be distinguished and identified based on the rate constants and heterogeneous and homogeneous electron and ion transfer rates.

The illustrated example embodiments will be best understood by reference to the figures. The following description is intended only by way of example, and simply illustrates certain example embodiments.

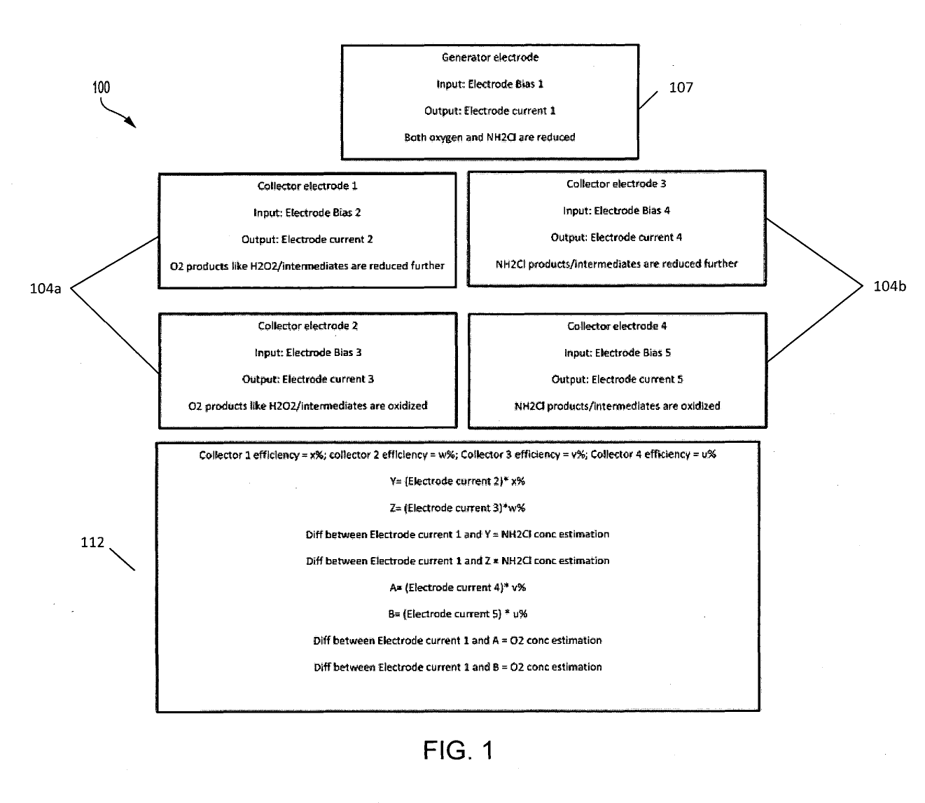

Referring now to FIG. 1, at 100, a schematic description of the forthcoming embodiments is presented, in particular, a first embodiment is directed to an electrode array in which at least two pairs of electrodes are used to distinguish and measure oxygen (O₂) and monochloramine (NH₂Cl) present in a sample. Each pair comprises a generator electrode and a collector electrode. There is also included in the arrangement one or more auxiliary and reference electrodes.

In an embodiment, the generator electrode 107 is set to electrochemically reduce both oxygen and monochloramine. The cathodic current at the generator electrodes arises from the simultaneous reduction of both oxygen and monochloramine species. The collector electrodes of the two sets are biased at different potentials. For example, the collectors 1 & 2 (indicated at 104a) assigned to the oxygen measurement may be biased at a potential where products from oxygen reduction at the generator electrode (such as hydrogen peroxide (H₂O₂)) are reduced. Collector electrodes 3 & 4 (indicated at 104b) are biased at a potential where the intermediates or products generated by the reduction of monochloramine are reduced or oxidized.

If, for instance, there are no electrochemically reactive intermediates or products produced by the reduction of monochloramine at the generator electrode 107, it is relatively easy to distinguish between oxygen and monochloramine. A collector current can be attributed to the formation of hydrogen peroxide or other intermediate species produced from oxygen reduction at the generator electrode 107. The magnitude of the signal obtained from the collector electrode is directly proportional to the oxygen present in the system. This signal can be used to quantify the concentration of oxygen in the sample. Subtraction of the collection electrode current (corrected for the collection efficiency) from the current response obtained from the generator electrode 107 for reduction of both oxygen and monochloramine provides a way to obtain the concentration of both oxygen and monochloramine in the sample

Redox active intermediates or products formed from the reduction of monochloramine at the generator electrode 107 can provide a signal different from that obtained from the oxygen reduction at the collector electrodes. In an embodiment, the collector electrodes assigned to oxygen and monochloramine are biased at different potentials (as indicated in FIG. 1) to monitor the electrochemical perturbation of the products/intermediates arising from these two species. Here, the differential in the signals arising from irreversible reactions obtained from the electrochemical perturbation of the products/intermediates between oxygen and monochloramine will provide data for distinguishing between the two species. The differential collector signal obtained between the oxygen products and monochloramine product redox reactions are used along with the combined reduction signal of oxygen and monochloramine obtained at the generator electrode to determine oxygen and monochloramine concentrations in a sample. For example, at 112, an estimate of the monochloramine concentration in a water sample may be determined by examining the difference between electrode current 1 and electrode current 2 as well as the difference between electrode current 1 and electrode current 3. An estimate of the oxygen concentration in a water sample may be determined by examining the difference between electrode current 1 and electrode current 4 as well as the difference between electrode current 1 and electrode current 5.

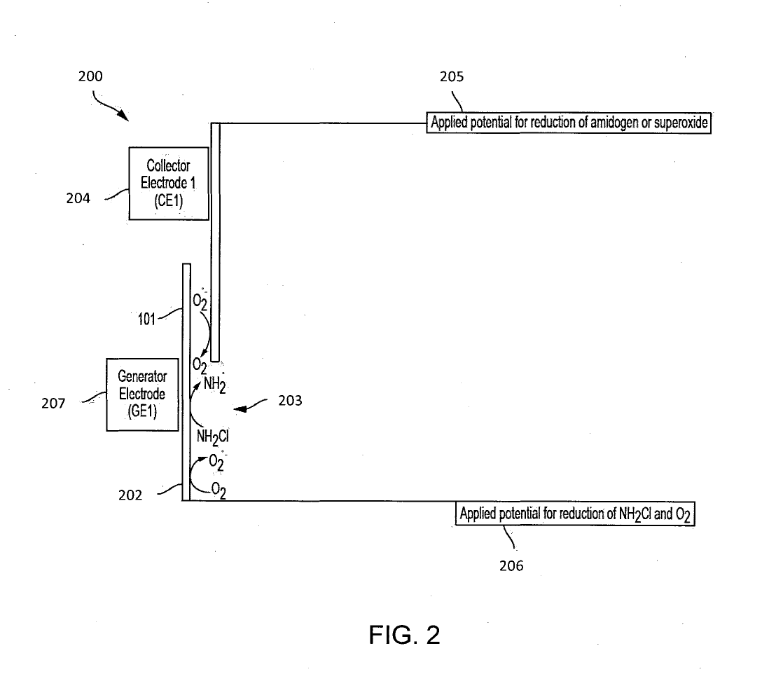

Referring now to FIG. 2, at 200, an electrode array for a direct detection method according to an embodiment is illustrated. Reduction of oxygen can occur via an electrochemical-chemical-electrochemical (E-C-E) process in aqueous media, e.g., water sample. In an embodiment, a first step is an electrochemical process in which the oxygen is reduced, at 202, to superoxide radical (O₂^{.-}) in a one electron transfer step. This superoxide radical may then react chemically with water molecules to produce hydrogen peroxide (H₂O₂). Hydrogen peroxide can also be reduced electrochemically at this same potential to hydroxide species.

Monochloramine can undergo an initial electrochemical reaction, at 203 to form an amidogen radical (NH₂^{.}), which can then react chemically with water to form an adduct that then chemically speciates into ammonia (NH₃) or ammonium hydroxide (NH₄OH). Under certain sample conditions, the amidogen radical can also dimerize or can be scavenged in the presence of carbonate. All these are chemical reactions that can occur after the initial reduction 203 of monochloramine. Hence, electrochemically reduced monochloramine undergoes reaction pathways significantly different from oxygen reduction.

In FIG. 2, a generator electrode (GE1) 207 and a collector electrode (CE1) 204 arranged adjacent to, but physically apart, from each other are illustrated. Monochloramine and oxygen undergo initial reduction at GE1 207. The products of these electrochemical reactions are carried or diffused to the downstream or adjacent CE1 204. The collector electrode, CE1 204, is held at a potential 205 that is sufficient to detect irreversible reaction products/processes like the superoxide or amidogen radical or hydrogen peroxide, for example, which are produced at GE1 207. The current 206 generated at GE1 207 for the reduction of monochloramine and oxygen will be compared with the current 205 generated at CE1 204 corresponding to superoxide/hydrogen peroxide and/or amidogen radical/adduct. A ratio metric analysis of these currents will be used to distinguish between the analyte of interest (monochloramine) and the interfering species (oxygen).

The advancement of microfabrication techniques allows precise nanometer or sub-nanometer spacing between generator electrode 207 and collector electrode 204, which allows the detection of short lived intermediates, such as superoxide or amidogen radical. Very fast pulse techniques and scan rates may be employed and allow the detection of these short lived intermediates at different potentials. The variables including but not limited to, the spacing of the electrodes, electrochemical perturbation techniques (pulsing/scan rates) etc., can also be leveraged to distinguish or prevent the cross reaction between the intermediates and/or reactants and/or products that are initially formed or that are formed during the electrochemical/chemical conversion of the analyte(s) and interferant(s) into other forms.

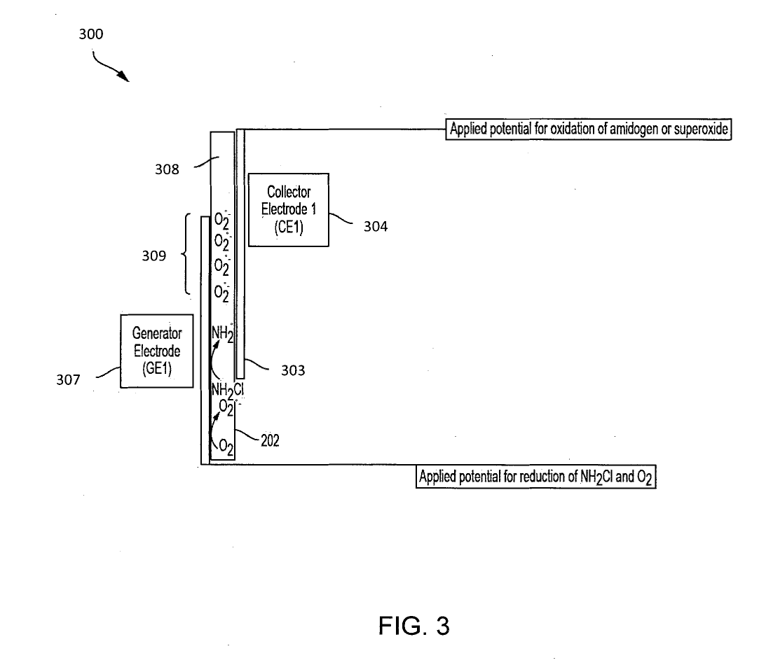

Referring now to FIG. 3, at 300, an electrode array for a spaced layer method is illustrated. Materials can be placed (by a suitable technique such as coating/adsorption/immobilization) in between the generator electrode 307 and collector electrode 304 for enabling selective reaction, stabilization, and/or adsorption of intermediates produced at the generator electrode 307. As an example, trivalent aluminum compounds are known to stabilize the superoxide radical 309. In an embodiment, a thin layer 308 of Al(III) compound deposited between the generator electrode 307 and collector electrode 304 stabilizes the superoxide radical 309, allowing the superoxide radical 309 to make the transit to the collector electrode 304 where it can be measured.

In an embodiment, a thin layer 308 that preferentially reacts with intermediates or by-products of the reduction 303 of monochloramine can similarly be placed in between the generator electrode 307 and collector electrode 304. This enables the characterization of the monochloraine reduction that leads to its discrimination from interfering species like oxygen, manganese, and iron. For example, the thin layer 308 can be a copper layer that can be deposited in between the generator electrode 307 and collector electrode 304, which reacts with the amine/ammonia species produced from the reduction of monochloramine at the generator electrode 307. Controlled fast pulse/scan potentials can be delivered to the copper layer placed in between the generator electrode 307 and collector electrode 304. Potentiometric methods enables measurement of the potential difference for the copper deposition/dissolution in the presence and absence of the intermediates/products and byproducts of the monochloramine reduction. This can be extended to other disinfectants and nutrients that produce intermediates, products, or byproducts that exhibit chemical or electrochemical reactions with specific materials that can be deposited in the region between a generator electrode 307 and collector electrode 304.

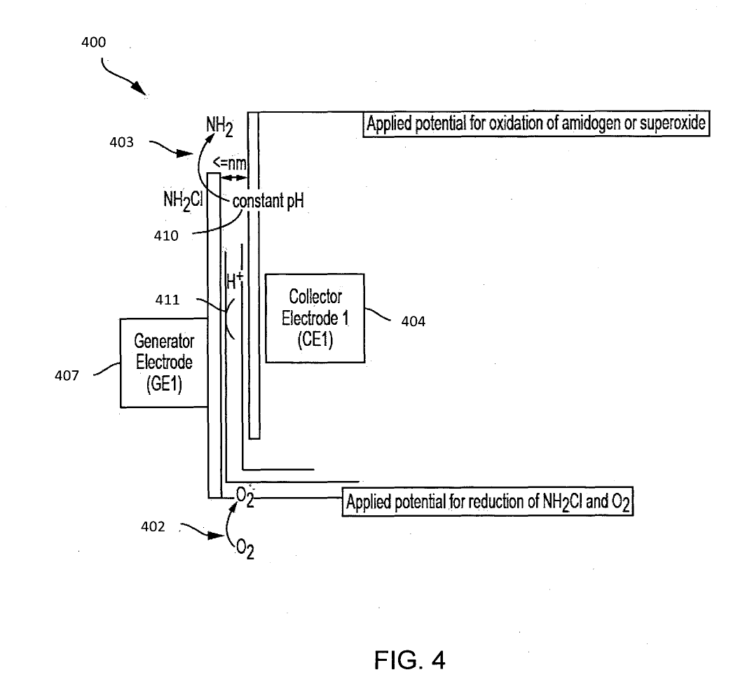

Referring now to FIG. 4, at 400, an electrode array for a pH control method is illustrated. The mechanistic reduction pathway of oxygen is significantly dependent on the pH. Oxygen reduction adopts the following reaction scheme:

O₂+e⁻=O₂^{-.}

O₂^{-.}+H⁺=HO^{2.}

HO₂^{.}+O₂^{-,}=HO₂⁻+O₂

HO₂⁻+H⁺=H₂O₂

H₂O₂+2e⁻+2H⁺=4H₂O

Overall Reaction: O₂+4H⁺+4e⁻=2H₂O

The chemical reduction of oxygen needs four protons whereas the monochloramine needs two protons: NH₂Cl+2e⁻+2H⁺=NH₄⁺+Cl⁻.

This pH dependence, especially in the pH in the vicinity of the electrodes 404, 407 affects the reaction pathway and kinetics of both oxygen and monochloramine reduction. A high density of proton-generating nanoelectrodes 411 can be packaged in a small unit area resulting in an efficient proton-generating system because of the high surface area, which can be used to produce a controlled, constant pH 410 as indicated. This pH altering method can be used to control the chemical reaction products of oxygen 402 and monochloramine reduction 403 (such as super oxide versus hydrogen peroxide formation or amidogen versus ammonia formation), thus facilitating discrimination of the reduction of oxygen from monochloramine in a generator-collector system. Modulating proton production in order to vary the stability of the intermediate/product formation that generates a redox response at the collector electrode 404 can provide for further discrimination of the two classes of the oxygen and monochloramine reduction intermediates/products/byproducts. That is, for example, activating and deactivating the proton formation at the generator electrode 407 and/or the collector electrode 404 in some pattern may provide for greater ability to differentiate between the analyte (monochloramine) and an interferant (such as oxygen).

In an embodiment, the measurement of chlorine in water by amperometry is achieved by the use of a bare noble metal or carbon electrode. The electrochemical reduction of chlorine (primarily as HOCl and OCl⁻) is pH dependent and the reduction of HOCl is more readily reduced than OCl⁻. The ratio of HOCl to OCl⁻ increases as pH decreases. Thus, as the pH increases, the reduction of chlorine decreases for modest reduction potentials; as is noted in most bare electrode, commercially available amperometric chlorine sensors.

For measurement of residual chlorine in water, the pH is typically controlled by means of pH modification to a certain value or range by addition of pH buffers or acids/bases. The benchmark colorimetric method based on N,N′-diethyl-p-phenylenediamine (DPD) chemistry utilizes a buffer reagent to set the sample pH to a certain value for optimal determination of the chlorine concentration in a sample. Common electrochemical methods employ buffers added to a sample or retained between a membrane and a measurement electrode in order to optimize the chlorine measurement. Another common approach for electrochemical methods is to employ an additional measurement sensor which measures pH (such as a glass ISE specific for pH measurement). The measured pH value is used to mathematically adjust or correct the measured chlorine value based upon the sample pH.

In an embodiment, measuring the sample pH without the need of an added pH sensor is presented. An embodiment does so without the addition of any reagents, buffers, or electrochemical pH modification. According to an embodiment, the measurement of chlorine and pH can be obtained and provide for a pH-corrected chlorine measurement by means of a standard 3-electrode configuration without a membrane or buffers/reagents.

For example, in an embodiment the pH of the sample may be determined by the reduction of the chlorine analyte itself. By scanning the voltage of the working electrode from a positive value where no reduction of chlorine occurs to a more negative voltage beyond which the reduction of chlorine occurs, a sigmoidal-type response for the reduction current is noted when micro- or nano-electrodes or controlled convection are employed. The inflection point potential for the reduction response (or other key response features such as onset potential of current response) can be correlated to the sample pH. The potential of the inflection point for the reduction of chlorine in water is noted to shift to more negative potentials as the pH increases. The onset potential, half-peak potential, and the peak potential will shift to more negative potentials with an increase in pH. Linear scan voltammetry can be utilized to obtain this response data. When employing linear scan voltammetry, the potential of the inflection point for the chlorine reduction wave is determined and provides a pH value; additionally, the peak current response for chlorine reduction in the same voltammogram can be used to determine the chlorine concentration. The measured chlorine value by the scan can be corrected with the pH measured by the inflection point potential; thereby providing a more accurate chlorine concentration value than that obtained without the pH correction. The inflection point or the half-peak potential dependence on the pH can be determined for a controlled system like a drinking water distribution system using the Nernst equation.

In an embodiment, cyclic voltammetry may also be used, as may several established pulsed voltammetry methods (e.g., DPV, SWV, etc.). Features, such as peak potentials or other reduction potential inflection points obtained in scans, may be used in a similar manner as above to obtain the desired pH and/or chlorine measurement. Changing the scan rate and examining the kinetics of the reduction may also provide information to improve the measurement of the sample pH by this method. Scan methods may provide both pH and chlorine values; however, an embodiment could also utilize the scanning or pulsed voltammetry to obtain pH and/or chlorine measurements while coupling with chronoamperometric methods for obtaining an additional, and perhaps more accurate, measure of the chlorine reduction which is then corrected by the measured pH value.

Factors that may affect this inflection point may include conductivity, presence of interferences in the sample, temperature, and electrode material. Since all these factors are fairly constant in the distribution system the inflection point primarily depends on the pH of the system.

The aforementioned embodiments of a pH control method provide a more accurate chlorine measurement with a simpler and more cost effective sensor than existing electrochemical probes. The lack of an added pH sensor and reagents allows a sensor, according to the aforementioned embodiments, to be more readily applicable to in-pipe applications for distribution monitoring, for example. Such implementation of a chlorine sensor is challenging for most existing probes on the market today.

In an embodiment, to discriminate the over potentials of oxygen and monochloramine, modifications to gold electrodes externally by polymers like poly 3,4 ethylene dioxy thiophene or internally through encapsulated chloride or other anions can be performed. This provides enhanced selectivity for monochloramine, especially in a nano-electrode configuration where the encapsulation or modification can be achieved with high accuracy. Therefore, by changing the internal or external composition of the gold nanostructures, the signals arising from monochloramine and oxygen can be differentiated. FIG. 4 is a schematic description, in particular to the step by step process of recording and calculation of the O₂ and NH₂Cl signals from differential measurements.

As used herein, the singular “a” and “an” may be construed as including the plural “one or more” unless clearly indicated otherwise.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. A method, comprising:
initiating, at a generator electrode in an electrode array having a collector electrode adjacent to but physically separate from the generator electrode, a reduction reaction for an oxygen containing species and a monochloramine species present in a water sample;
said initiating comprising generating, at the generator electrode, a generator current producing the reduction reaction;
detecting, at the collector electrode, a collector current associated with products formed from the reduction reaction;
wherein the electrode array is biased to preferentially detect one or more products of the reduction reaction; and
determining, by comparing the generator current with the collector current, concentrations of oxygen containing species and monochloramine species present in the water sample.

2. The method of claim 1, wherein the electrode array is biased by placing the collector electrode at a predetermined potential.

3. The method of claim 2, wherein the collector electrode preferentially detects at least one of the one or more products based upon the predetermined potential.

4. The method of claim 1, wherein the electrode array is biased by controlling a pH value of a solution proximate to one or more of the generator electrode and the collector electrode.

5. The method of claim 4, wherein the pH value of the solution influences sensitivity of one or more of the generator electrode and the collector electrode to reduction reaction products.

6. The method of claim 1, wherein the electrode array is biased using a thin layer of material located between the generator electrode and the collector electrode.

7. The method of claim 6, wherein the using the thin layer of material comprises providing an Al(III) compound layer that stabilizes a superoxide radical during transit to the collector electrode.

8. The method of claim 6, wherein the using the thin layer of material comprises providing a copper layer that preferentially reacts with products of the monochloramine species of the reduction reaction.

9. The method of claim 1, wherein the determining comprises utilizing ratio metric analysis.

10. An apparatus, comprising:
an electrode array, comprising:
a generator electrode; and
a collector electrode;
said electrode array having the collector electrode disposed adjacent to but physically separate from the generator electrode;
the generator electrode providing a current that initiates a reduction reaction for an oxygen containing species and a monochloramine species present in a water sample;
the collector electrode detecting a collector current associated with products formed from the reduction reaction;
wherein the electrode array is biased to preferentially detect one or more products of the reduction reaction; and
a processor that determines, by comparing the generator current with the collector current, concentrations of oxygen containing species and monochloramine species present in the water sample.

11. The apparatus of claim 10, wherein the electrode array is biased by placing the collector electrode at a predetermined potential.

12. The apparatus of claim 11, wherein the collector electrode preferentially detects at least one of the one or more products based upon the predetermined potential.

13. The apparatus of claim 10, comprising one or more proton generating nanotubes, wherein the electrode array is biased by controlling a pH value of a solution proximate to one or more of the generator electrode and the collector electrode.

14. The apparatus of claim 13, wherein the pH value of the solution influences sensitivity of one or more of the generator electrode and the collector electrode to reduction reaction products.

15. The apparatus of claim 10, comprising a thin layer of material, wherein the electrode array is biased using the thin layer of material located between the generator electrode and the collector electrode.

16. The apparatus of claim 15, wherein the thin layer of material comprises an Al(III) compound layer that stabilizes a superoxide radical during transit to the collector electrode.

17. The apparatus of claim 15, wherein the thin layer of material comprises a copper layer that preferentially reacts with products of the monochloramine species of reduction reaction.

18. The apparatus of claim 10, wherein the processor utilizes ratio metric analysis to determine, by comparing the generator current with the collector current, concentrations of oxygen containing species and monochloramine species present in the water sample.

19. The apparatus of claim 10, wherein the electrode array comprises a plurality of collector electrodes, wherein each of the plurality of collector electrodes is biased at a predetermined potential.

20. A chlorine probe, comprising:
an electrode array, comprising:
a metal generator electrode; and
a metal collector electrode;
said electrode array having the collector electrode and the generator electrode deposited as nanostructures adjacent to, but physically separate from, one another;
the generator electrode:
providing a current that initiates a reduction reaction for an oxygen containing species and a monochloramine species present in a water sample;
the collector electrode detecting a collector current associated with products, byproducts or intermediate species formed from the reduction reaction;
wherein the electrode array is biased to preferentially detect the one or more products byproducts or intermediate species of the reduction reaction by use of a biasing mechanism selected from the group consisting of:
a thin layer of material deposited between the metal generator electrode and the metal collector electrode that preferentially absorbs or stabilizes one or more of the products, byproducts or intermediate species; and
a pH control element that generates protons between the metal generator electrode and the metal collector electrode; and
a processor that determines, by comparing the generator current with the collector current, concentrations of oxygen containing species and monochloramine species present in the water sample.
